(19) United States
(12) Patent Application Publication
(10) Pub. No.: US 2006/0281390 A1
(43) Pub. Date: Dec. 14, 2006

(54) Mastectomy bras

(76) Inventor: Chang-Yu Lin, Chang-Hua (TW)

Correspondence Address: LIN, CHANG-YU, P.O. BOX 487, CHANG-HUA CITY, 500, TW

(21) Appl. No.: 11/144,774

(22) Filed: Jun. 6, 2005

(51) Int. Cl. A41C 3/10; A61F 2/52

(52) U.S. Cl. 450/57; 623/7

(57) ABSTRACT

A mastectomy bra includes two cups and one of the cups has an inner layer and an outer layer which is sewed to an inside of the outer layer. A resilient support member is fixedly connected between the inner layer and the outer layer. A mammary prosthesis which is a bladder with silica gel and whiteoil received therein and the bladder is located between the inner layer and the outer layer. The resilient support member is located beneath and supporting the mammary prosthesis. The mammary prosthesis is well positioned and has a soft touch which makes the wearers feel comfortable.

### FIELD OF THE INVENTION

The present invention relates to a mastectomy bra having a soft bladder as a mammary prosthesis fixed in at least one of the two cups of the bra.

### BACKGROUND OF THE INVENTION

A conventional mastectomy bra generally includes at least one mammary prosthesis located in one of the two cups of the bra and the mammary prosthesis includes an inside with smaller curvature which is expected to match with the user's skin and an outside with a larger curvature which looks the same as the curvature of the other cup. However, the mammary prosthesis is not well positioned in the cup so that it might drop when wearing or taking off the bra. The mammary prosthesis generally is made by Ethylene-Vinyl Acetate Resin Emulsion which is not an air-permeable product so that the user feel not comfortable when wearing it in hot summer. Besides, due to gravity, the mammary prosthesis presses on the surgery portion of the users.

A main object of the present invention is to provide a mastectomy bra which includes at least one mammary prosthesis which is fixed between an inner layer and an outer layer of the cup so that the users feel comfortable.

Another object of the present invention is to provide a mastectomy bra that includes a mammary prosthesis of a soft bladder which provide good touch with the users' skin.

Yet another object of the present invention is to provide a mastectomy bra that includes a decoration piece between the two cups so as to hide the scars of the surgery portion of the users.

### SUMMARY OF THE INVENTION

The present invention relates to a mastectomy bra that comprises two cups and each cup has an elastic side warp extending therefrom. One of the cups has an inner layer and an outer layer which is sewed to an inside of the outer layer. A resilient support member is fixedly connected between the inner layer and the outer layer. A mammary prosthesis is located between the inner layer and the outer layer. The resilient support member is located beneath and supporting the mammary prosthesis.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the mastectomy bra of the present invention;

FIG. 2 shows the components of the mastectomy bra of the present invention;

FIG. 3 is an exploded view to show one of the cups that includes a resilient support member and a mammary prosthesis;

FIG. 4 shows the cup with the resilient support member and the mammary prosthesis;

FIG. 5 shows a top view of the cup in FIG. 4.

### DETAILED DESCRIPTION OF THE PREFERRED EMBODIMENT

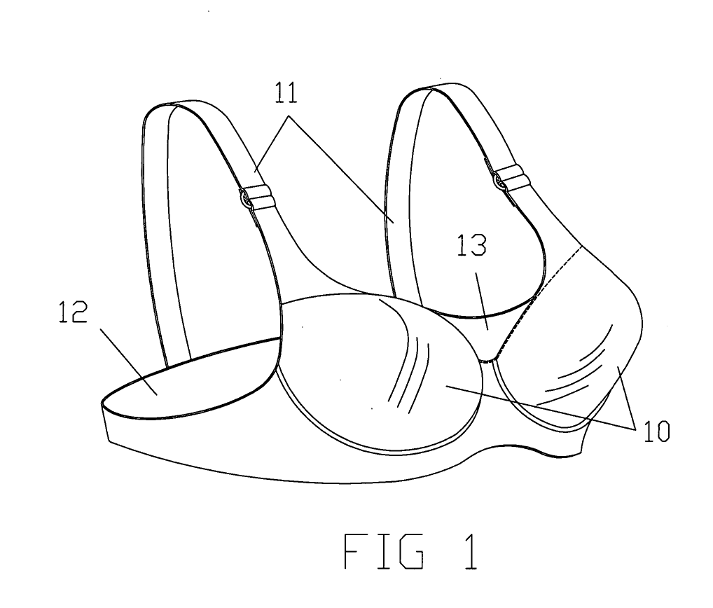

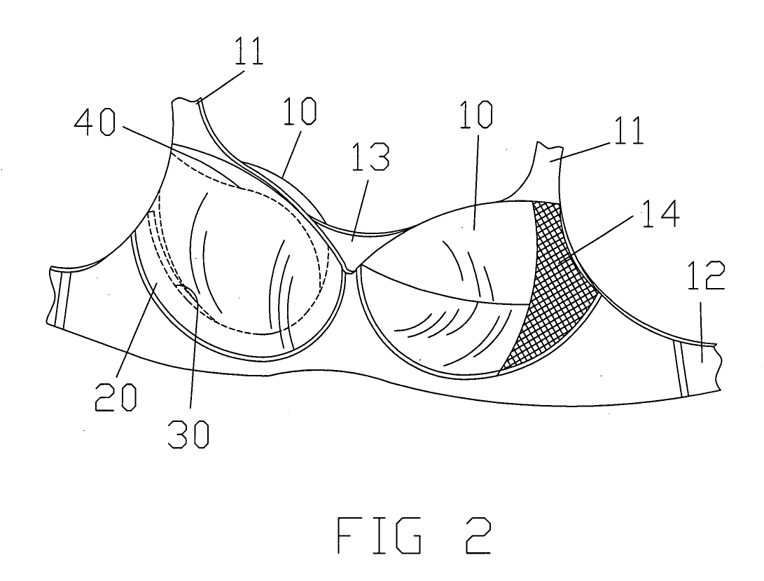

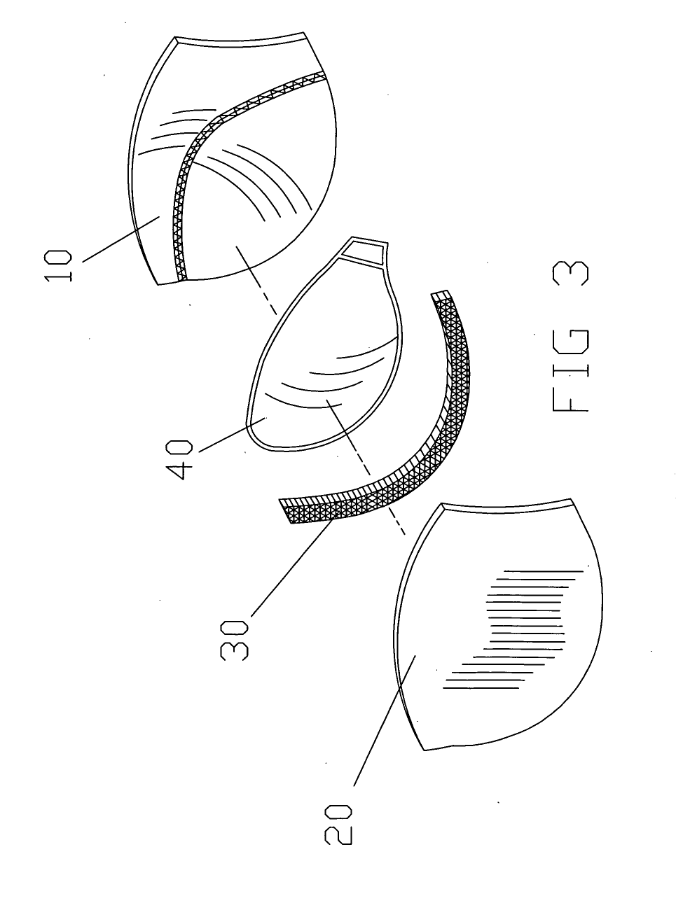

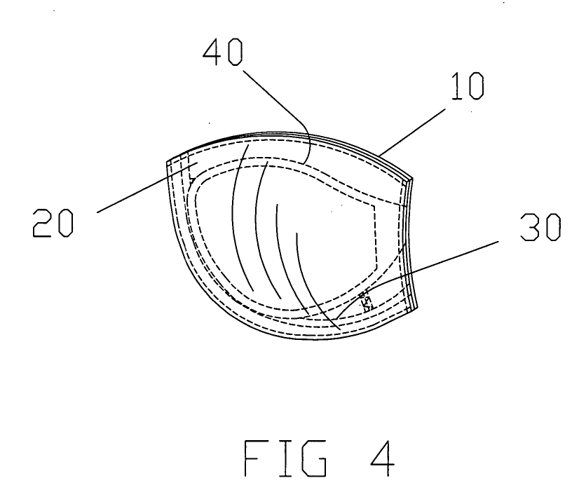

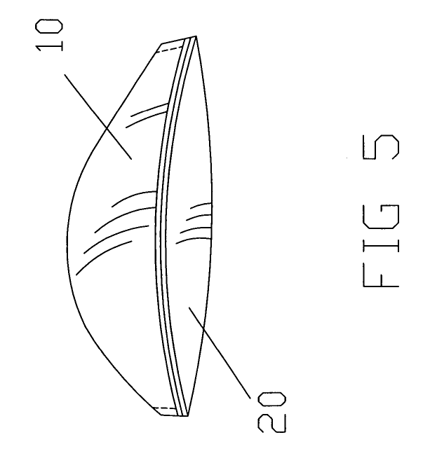

Referring to FIGS. 1 to 5, the mastectomy bra of the present invention comprises two cups 10 and each cup 10 having an elastic side warp 12 extending therefrom and two shoulder straps 11 are connected between the two cups 10 and the side wraps 12.

One of the cups 10 has an inner layer and an outer layer 20 which is sewed to an inside of the outer layer. Both of the inner layer and the outer layer 20 are made by air-permeable material such as silk cotton. A resilient support member 30 is fixedly connected between the inner layer and the outer layer 20 and is located at a lower portion of the cup 10. A mammary prosthesis 40 is located between the inner layer and the outer layer 20, the resilient support member 30 is located beneath and supporting the mammary prosthesis 40. The mammary prosthesis 40 is well positioned between the inner layer and the outer layer 20 so that the wearers do not worry about shifting or falling of the mammary prosthesis 40.

The mammary prosthesis 40 is a bladder in which a mixture of silica gel and whiteoil is received. The silica gel and the whiteoil are mixed by a pre-decided ratio to make the bladder have soft touch and will not stimulate the surgery portion of the wearers.

For the other cup 10, a non-flex netted fabric 14 is fixed connected between the other cup 10 and the side wrap 12 of said the other cup 10 and the non-flex netted fabric 14 provides a comfortable support to the breast.

A decoration piece 13 is connected between two respective top edges of the two cups 10 so that scars of surgery of the wearers can be hidden by the decoration piece 13.

While we have shown and described the embodiment in accordance with the present invention, it should be clear to those skilled in the art that further embodiments may be made without departing from the scope of the present invention.

What is claimed is:

1. A mastectomy bra comprising:
two cups and each cup having an elastic side warp extending therefrom, one of the cups having an inner layer and an outer layer which is sewed to an inside of the outer layer, a resilient support member fixedly connected between the inner layer and the outer layer, and
a mammary prosthesis located between the inner layer and the outer layer, the resilient support member located beneath and supporting the mammary prosthesis.

2. The mastectomy bra as claimed in claim 1, wherein the mammary prosthesis is a bladder in which silica gel and whiteoil are received.

3. The mastectomy bra as claimed in claim 1, wherein a decoration piece is connected between two respective top edges of the two cups.

4. The mastectomy bra as claimed in claim 1, wherein a non-flex netted fabric is fixed connected between the other cup and the side wrap of said the other cup.

5. The mastectomy bra as claimed in claim 1, wherein the inner layer of each of the two cups is made by air-permeable material.

6. The mastectomy bra as claimed in claim 5, wherein the air-permeable material is silk cotton.

7. The mastectomy bra as claimed in claim 1, wherein the outer layer of each of the two cups is made by air-permeable material.

8. The mastectomy bra as claimed in claim 7, wherein the air-permeable material is silk cotton.
